(19) United States
(12) Patent Application Publication
(10) Pub. No.: US 2024/0082424 A1
(43) Pub. Date: Mar. 14, 2024

(54) CLATHRATE IN WHICH EQUOL IS INCLUDED IN CYCLODEXTRIN, EQUOL-ABSORBENT COMPOSITION CONTAINING SAID CLATHRATE, AND PRODUCTION METHOD THEREFOR

(71) Applicant: DAICEL CORPORATION, Osaka-shi, Osaka (JP)

(72) Inventor: Motoko HAYASHI, Tokyo (JP)

(73) Assignee: DAICEL CORPORATION, Osaka-shi, Osaka (JP)

(21) Appl. No.: 18/269,390

(22) PCT Filed: Dec. 24, 2021
(86) PCT No.: PCT/JP2021/048167

§ 371 Date: Jun. 23, 2023

(30) Foreign Application Priority Data
Dec. 24, 2020 (JP) 2020-215247

(51) Int. Cl. A61K 47/69; A61K 9/00

(52) U.S. Cl. CPC A61K 47/6951; A61K 9/0053

(57) ABSTRACT

The present invention provides a clathrate in which equol is included in a cyclodextrin, and an equol-absorbent composition containing an equol clathrate in which equol is included in a cyclodextrin. In addition, the present invention provides a method for producing the clathrate or the equol-absorbent composition, the method including adding a cyclodextrin to a fermentation medium during a fermentative production of equol from at least one equol raw material selected from the group consisting of daidzein glycosides, daidzein, and dihydrodaidzein, using a microorganism that assimilates the equol raw material into equol.

### TECHNICAL FIELD

The present invention relates to a clathrate in which equol is included in a cyclodextrin, and an equol-absorbent composition containing the clathrate. The present invention also relates to a method for producing the clathrate and a method for producing the equol-absorbent composition.

### BACKGROUND ART

Equol exerts female hormone actions (that of estrogen) and antioxidant actions, and is expected to have a preventive effect on breast cancer, prostate cancer, osteoporosis, hypercholesterolemia, heart disease, menopausal disorder, and the like. Equol is also sold as a health food for ameliorating these conditions and the like.

Equol sold as a health food is provided as tablets, and the dose thereof is about from 3 to 4 tablets, or from 1000 to 4000 mg, per day. The size of the tablets varies depending on the manufacturer. When the dose per tablet is large, the size of the tablets increases, which may make some people feel that the tablets are difficult to swallow at the time of administration. In addition, the dose also increases, which is in itself a problem.

Therefore, there is a demand for equol-containing tablets and the like that can improve the absorption or migration of equol into blood, reduce the dose per tablet or other forms, and reduce the size of the tablets or other forms.

### RELATED ART DOCUMENTS

### Patent Documents

Patent Document 1: JP H9-309902 A
Patent Document 2: JP 2012-77087 A
Patent Document 3: JP 2010-24240 A
Patent Document 4: JP 2010-187647 A
Patent Document 5: JP 5851686 B

### SUMMARY OF THE INVENTION

### Problems to be Solved by the Invention

The present invention has been made in view of the above-mentioned needs, and an object of the present invention is to provide an equol clathrate having improved solubility in water for achieving improved absorption or migration of equol into blood, and an equol-absorbent composition containing the equol clathrate. In particular, an object of the present invention is to provide an equol clathrate that can further increase the concentration of equol in blood when orally ingested as a food or the like, and an equol-absorbent composition containing the equol clathrate.

Aside from or in addition to the above objects, another object of the present invention is to provide a method for producing an equol clathrate having improved solubility in water and a method for producing an equol-absorbent composition containing the equol clathrate.

### Means for Solving the Problems

In order to solve the above problems, the present inventors have devised the following inventions.
(1) A clathrate in which an equol is included in a cyclodextrin.
(2) An equol-absorbent composition containing an equol clathrate in which an equol is included in a cyclodextrin.
(3) In (2) above, the composition may be of one type selected from the group consisting of a food composition and a pharmaceutical composition.
(4) In (2) or (3) above, a solubility of the equol or the equol clathrate in water may be 0.5 mg/mL or greater, preferably 1.0 mg/mL or greater, and more preferably 1.5 mg/mL or greater.
(5) In any one of (1) to (4) above, the cyclodextrin is preferably of one or more types selected from the group consisting of α-cyclodextrin, β-cyclodextrin, γ-cyclodextrin, and derivatives of the foregoing, preferably of one or more types selected from the group consisting of α-cyclodextrin, β-cyclodextrin, γ-cyclodextrin, glycosyl-β-cyclodextrin, maltosyl-β-cyclodextrin, hydroxypropyl-β-cyclodextrin, and methyl-β-cyclodextrin, more preferably of one or more types selected from the group consisting of β-cyclodextrin, glycosyl-β-cyclodextrin, maltosyl-β-cyclodextrin, hydroxypropyl-β-cyclodextrin, and methyl-β-cyclodextrin, and most preferably β-cyclodextrin.
(6) The cyclodextrin may be contained in an amount of 0.5 to 5000 parts by weight, preferably 1 to 100 parts by weight, more preferably 3 to 10 parts by weight, relative to 1 part by weight of the equol.
(7) A method for producing a clathrate in which an equol is included in a cyclodextrin, the method including adding a cyclodextrin to a fermentation medium during a fermentative production of equol from at least one type of equol raw material selected from the group consisting of daidzein glycosides, daidzein, and dihydrodaidzein, using a microorganism that assimilates the equol raw material into equol.
(8) A method for producing an equol-absorbent composition containing a clathrate in which equol is included in a cyclodextrin, the method including adding a cyclodextrin to a fermentation medium during a fermentative production of equol from at least one type of equol raw material selected from the group consisting of daidzein glycosides, daidzein, and dihydrodaidzein, using a microorganism that assimilates the equol raw material into equol.
(9) In (8) above, the method preferably further includes adding an orally ingestible substance to produce an equol-absorbent composition containing an equol clathrate in which equol is included in a cyclodextrin.
(10) Any one of (7) to (9) above may further include adding a cyclodextrin after the fermentative production process.
(11) In any one of (8) to (10) above, the composition is preferably of one type selected from the group consisting of a food composition and a pharmaceutical composition.
(12) In any one of (7) to (11) above, the microorganism that assimilates the equol raw material into equol is preferably an anaerobic microorganism.
(13) In any one of (7) to (12) above, the anaerobic microorganism may be of at least one type selected from the group consisting of microorganisms of the genus Adlercreutzia.
(14) In any one of (7) to (13) above, a solubility of equol or the equol clathrate in water may be 0.5 mg/mL or greater, preferably 1.0 mg/mL or greater, and more preferably 1.5 mg/mL or greater.
(15) In any one of (7) to (14) above, the cyclodextrin is preferably of one or more types selected from the group consisting of α-cyclodextrin, β-cyclodextrin, γ-cyclodextrin, and derivatives of the foregoing, preferably of one or more types selected from the group consisting of α-cyclodextrin, β-cyclodextrin, γ-cyclodextrin, glycosyl-β-cyclodextrin, maltosyl-β-cyclodextrin, hydroxypropyl-β-cyclodextrin, and methyl-β-cyclodextrin, more preferably of one or more types selected from the group consisting of β-cyclodextrin, glycosyl-β-cyclodextrin, maltosyl-β-cyclodextrin, hydroxypropyl-β-cyclodextrin, and methyl-β-cyclodextrin, and most preferably β-cyclodextrin.
(16) In any one of (7) to (15) above, the cyclodextrin may be added in an amount of 0.5 to 5000 parts by weight, preferably 1 to 100 parts by weight, more preferably 3 to 10 parts by weight, relative to 1 part by weight of equol.

### Effects of the Invention

The present invention can provide an equol clathrate having improved solubility in water for achieving improved absorption or migration of equol into blood, and an equol-absorbent composition containing the equol clathrate. In particular, the present invention can provide an equol clathrate that can further increase the concentration of equol in blood when orally ingested as a food or the like, and an equol-absorbent composition containing the equol clathrate.

Aside from or in addition to the above effects, the present invention can provide a method for producing an equol clathrate having improved solubility in water and a method for producing an equol-absorbent composition containing the equol clathrate.

### DESCRIPTION OF EMBODIMENTS

The present application provides an equol clathrate and an equol-absorbent composition containing the equol clathrate, as well as a method for producing the equol clathrate and a method for producing the equol-absorbent composition. The description is as follows.

### <Equol Clathrate>

The present application provides an equol clathrate, that is, a clathrate in which an equol is included in a cyclodextrin.

The cyclodextrin here is preferably of one or more types selected from the group consisting of α-cyclodextrin, β-cyclodextrin, γ-cyclodextrin, and derivatives of the foregoing.

The cyclodextrin is preferably of one or more types selected from the group consisting of α-cyclodextrin, β-cyclodextrin, γ-cyclodextrin, glycosyl-β-cyclodextrin, maltosyl-β-cyclodextrin, hydroxypropyl-β-cyclodextrin, and methyl-β-cyclodextrin.

The cyclodextrin is more preferably of one or more types selected from the group consisting of β-cyclodextrin, glycosyl-β-cyclodextrin, maltosyl-β-cyclodextrin, hydroxypropyl-β-cyclodextrin, and methyl-β-cyclodextrin.

The cyclodextrin is most preferably β-cyclodextrin.

The total amount of the cyclodextrin in the clathrate according to an embodiment of the present invention may be from 0.5 to 5000 parts by weight, preferably from 1 to 100 parts by weight, more preferably from 3 to 10 parts by weight relative to 1 part by weight of the total amount of equol.

### <Equol-absorbent Composition Containing Equol Clathrate>

The present application also provides an equol-absorbent composition containing the equol clathrate, i.e., an equol-absorbent composition containing the equol clathrate in which equol is included in a cyclodextrin.

The cyclodextrin and the equol clathrate have the same definitions as described above.

The equol-absorbent composition may contain, in addition to the equol clathrate, free cyclodextrin, free equol, and other components.

Examples of other components include, but are not limited to, medium components and metabolites thereof used in the production method to be described below, fermented products produced by the fermentation to be described below or a part of the fermented products, sugars removed from daidzein glycosides, enzymes used for treating glycosides, isoflavones other than daidzein compounds (including glycosides and isoflavone metabolites), and orally ingestible substances to be described below. In addition, the equol-absorbent composition may contain a cyclodextrin, other excipients, and the like added after completion of the fermentation.

The equol-absorbent composition according to an embodiment of the present application may have a solubility of equol or the equol clathrate in water of 0.5 mg/mL or greater, preferably 1.0 mg/mL or greater, and more preferably 1.5 mg/mL or greater. Note that, the upper limit of the solubility is preferably 11 mg/mL.

The equol-absorbent composition according to an embodiment of the present application may be of one type selected from the group consisting of a food composition, such as a health food composition, and a pharmaceutical composition, but is not limited thereto.

The form of the equol-absorbent composition according to an embodiment of the present application is not limited, and may be, for example, an oral formulation such as powder, tablets, coated tablets, granules, or capsules in consideration of oral ingestion.

The equol clathrate or the equol-absorbent composition containing the clathrate according to an embodiment of the present application has high absorbability and solubility of equol in water. As such, when equol clathrate or the equol-absorbent composition containing the clathrate according to an embodiment of the present application is made into the form of an oral formulation such as tablets, coated tablets, and capsules, equol can be absorbed at a desired concentration upon ingestion even with the reduced amount of equol per tablet. Thus, the amount of equol per tablet can be reduced to shrink the size of the tablets and the like.

When provided as a food composition, the equol clathrate or the equol-absorbent composition containing the clathrate according to an embodiment of the present application can be provided as an ingredient of food and beverages (including supplements).

Also, when provided as a food composition, the equol clathrate or the equol-absorbent composition containing the clathrate according to an embodiment of the present application can be used both as a general food and as a food for specified health uses, a nutritional supplement, a functional food, a food for the sick, a food additive, or the like. Examples of the form of foods include, but are not limited to, soft drink, milk, pudding, jelly, candy, gum, gummy, yogurt, chocolate, soup, cookie, snack, ice cream, ice pop, bread, cake, cream puff, ham, meat sauce, curry, stew, cheese, butter, dressing, supplement, food and drink for specific health uses, and energy drink that contain the equol-containing food composition according to an embodiment of the present invention.

The food composition may contain an orally ingestible substance. Examples of the orally ingestible substance include, but are not limited to, water, a protein, a carbohydrate, a lipid, a vitamin, a mineral, an organic acid, an organic base, a fruit juice, a flavor, and the like.

Examples of the protein include, but are not limited to, animal and vegetable proteins, such as whole milk powder, skimmed milk powder, partially skimmed milk powder, casein, soy protein, chicken egg protein, and meat proteins; hydrolysates of the foregoing; and butter.

Examples of the carbohydrate include, but are not limited to, sugars, processed starches (dextrin, as well as soluble starch, British starch, oxidized starch, starch ester, starch ether, and the like), and dietary fibers.

Examples of the lipid include, but are not limited to, animal oils, such as lard and fish oils, as well as fractionated oils, hydrogenated oils and transesterified oils of the foregoing; and vegetable oils, such as palm oil, safflower oil, corn oil, rapeseed oil, coconut oil, as well as fractionated oils, hydrogenated oils and transesterified oils of the foregoing.

Examples of the vitamin include, but are not limited to, vitamin A, carotenes, B-group vitamins, vitamin C, D-group vitamins, vitamin E, K-group vitamins, vitamin P, vitamin Q, niacin, nicotinic acid, pantothenic acid, biotin, inositol, choline, and folic acid.

Examples of the mineral include, but are not limited to, calcium, potassium, magnesium, sodium, copper, iron, manganese, zinc, selenium, and whey minerals.

These components can be used in combination of two or more, and synthetic products and/or foods containing these components in large amounts may be used.

The mixing ratio of the equol-absorbent composition in these foods can be appropriately set depending on the type of food, the content of equol, the age and sex of the person ingesting the food, the expected effect, and the like. The mixing ratio of the equol-absorbent composition may be, but is not limited to, 0.01 to 100 g, preferably 0.1 to 10 g, more preferably 0.5 to 5 g of equol or equol clathrate per 100 g of food. The daily intake of the food containing the equol-containing food composition varies depending on the content of equol in the composition, the age and weight of the person ingesting the food, the number of times of intake, and the like. For example, the daily intake may be an amount equivalent to 0.01 to 10 g of the composition per adult per day.

### <Method for Producing Equol Clathrate> and <Method for Producing Equol-Absorbent Composition Containing Equol Clathrate>

The present application provides a method for producing the equol clathrate described above. The present application also provides a method for producing an equol-absorbent composition containing the equol clathrate.

For example, the equol clathrate described above can be produced by the following method.

That is, the equol clathrate can be produced by adding a cyclodextrin to a fermentation medium during a fermentative production of equol from at least one equol raw material selected from the group consisting of daidzein glycosides, daidzein, and dihydrodaidzein, using a microorganism that assimilates the equol raw material into equol.

Further, for example, the equol-absorbent composition containing the equol clathrate can be produced by the following method.

That is, the equol-absorbent composition containing the equol clathrate in which an equol is included in a cyclodextrin can be produced by adding a cyclodextrin to a fermentation medium during a fermentative production of equol from at least one type of equol raw material selected from the group consisting of daidzein glycosides, daidzein, and dihydrodaidzein, using a microorganism that assimilates the equol raw material into equol.

Note that, in addition to being added to the “fermentation medium”, a cyclodextrin may be additionally added after the fermentative production process.

### <<Equol Raw Material>>

The equol raw material used in the production method according to an embodiment of the present invention may be in any form as long as it is literally used as a raw material of equol.

The equol raw material may be of at least one type selected from the group consisting of daidzein glycosides, daidzein, and dihydrodaidzein, and may be in any form. For example, the equol raw material may be a daidzein glycoside itself, daidzein itself, or dihydrodaidzein itself; or, the equol raw material may be a raw material containing a daidzein glycoside, daidzein, or dihydrodaidzein, with examples being soybean, processed soybean products, soybean hypocotyl, and processed soybean hypocotyl products such as soybean hypocotyl extract, specifically, commercially available isoflavones.

### <<Equal-assimilating Microorganism>>

The equol-assimilating microorganism used in the production method according to an embodiment of the present invention is not limited as long as it produces equol from the equol raw material described above.

Examples of the equol-assimilating microorganism include an anaerobic microorganism and lactic acid bacteria.

Note that, equol assimilation can be confirmed by quantifying daidzein, dihydrodaidzein, equol, and the like in the culture. Quantification of these chemicals can be carried out by those skilled in the art based on the descriptions of, for example, WO 2012/033150, JP 2012-135217 A, JP 2012-135218 A, JP 2012-135219 A, and the like. An example of a method for quantifying these chemicals is described below.

For example, ethyl acetate is added to a culture solution, vigorously stirred, and centrifuged. Then, the ethyl acetate layer is collected. The same operation can be performed several times as necessary using the culture solution, and the collected ethyl acetate layers can be combined to prepare a liquid extract of equol. The liquid extract is concentrated under reduced pressure using an evaporator, dried, and dissolved in methanol. The resulting liquid is filtered using a membrane such as a polytetrafluoroethylene (PTFE) membrane to remove the insoluble matter. The resulting product can be used as a measurement sample for high performance liquid chromatography. Examples of conditions for high performance liquid chromatography include, but are not limited to, the following.

### [Conditions for High Performance Liquid Chromatography]

Column: Phenomenox Luna 5u C18, 2.0 mm×150 mm (Shimadzu GLC)
Mobile phase: water/methanol [55:45, v/v]
Flow rate: 0.2 mL/min
Column temperature: 40° C.
Detection: UV (280 nm)
Retention time: 13.8 min for dihydrodaidzein, 19.6 min for daidzein, 22.5 min for glycitein, 25.6 min for equol, 35.0 min for genistein

Examples of the equol-assimilating microorganism include, but are not limited to, microorganisms classified into the following genera.
Genus Adlercreutzia
Genus Bacteroides
Genus Bifidobacterium
Genus Clostridium
Genus Eggerthella
Genus Enterococcus
Genus Enterorhabdus
Genus Eubacterium
Genus Finegoldia
Genus Lactobacillus
Genus Lactococcus
Genus Paraeggerthella
Genus Pediococcus
Genus Proteus
Genus Sharpea
Genus Slackia
Genus Streptococcus
Genus Veillonella

Specific examples of the equol-assimilating microorganism include, but are not limited to, the following microorganisms.
Adlercreutzia equolifaciens subsp. celatus
Adlercreutzia equolifaciens subsp. equolifaciens
Bacteroides ovatus
Bifidobacterium breve
Bifidobacterium longum
Clostridium sp.
Eggerthella sp.
Enterococcus faecalis
Enterococcus faecium
Enterorhabdus mucosicola
Eubacterium sp.
Finegoldia magna
Lactobacillus fermentum
Lactobacillus mucosae
Lactobacillus paracasei
Lactobacillus plantarum
Lactobacillus rhamnosus
Lactobacillus sp.
Lactococcus garvieae
Paraeggerthella sp.
Pediococcus pentosaceus
Proteus mirabilis
Sharpea azabuensis
Slackia equolifaciens
Slackia isoflavoniconvertens
Slackia sp.
Streptococcus constellatus
Streptococcus intermedius
Veillonella sp.

Among the microorganisms described above, the equol-assimilating microorganism may be, for example, a microorganism classified under the family Eggerthellaceae, a microorganism classified under the family Bifidobacteriaceae, a microorganism classified under the family Clostridiaceae, a microorganism classified under the family Coriobacteriaceae, a microorganism classified under the family Enterococcaceae, a microorganism classified under the family Eubacteriaceae, a microorganism classified under the family Morganellaceae, a microorganism classified under the family Peptoniphilaceae, a microorganism classified under the family Lactobacillaceae, a microorganism classified under the family Streptococcaceae, a microorganism classified under the family Veillonellaceae, or a microorganism similar or otherwise related to the foregoing. The equol-assimilating microorganism is preferably, a microorganism classified under the genus Adlercreutzia, the genus Bacteroides, the genus Bifidobacterium, the genus Clostridium, the genus Coriobacterium, the genus Eggerthella, the genus Enterococcus, the genus Eubacterium, the genus Finegoldia, the genus Lactobacillus, the genus Paraeggerthella, the genus Pediococcus, the genus Proteus, the genus Sharpea, the genus Slackia, the genus Streptococcus, the genus Veillonella, or a microorganism similar or otherwise related to the foregoing. The equol-assimilating microorganism is more preferably Adlercreutzia equolifaciens subsp. celatus, Adlercreutzia equolifaciens subsp. equolifaciens, Bacteroides ovatus, Bifidobacterium breve, Bifidobacterium longum, Clostridium sp., Eggerthella sp., Enterococcus faecalis, Enterococcus faecium, Enterorhabdus mucosicola, Eubacterium sp., Finegoldia magna, Lactobacillus fermentum, Lactobacillus intestinalis, Lactobacillus mucosae, Lactobacillus paracasei, Lactobacillus plantarum, Lactobacillus rhamnosus, Lactobacillus sp., Lactococcus garvieae, Paraeggerthella sp., Pediococcus pentosaceus, Proteus mirabilis, Sharpea azabuensis, Slackia equolifaciens, Slackia isoflavoniconvertens, Slackia sp., Streptococcus constellatus, Streptococcus intermedius, or Veillonella sp.

In particular, among the microorganisms described above, examples of the more preferred anaerobic microorganism include any of the microorganisms described below, or similar or otherwise related bacteria having species properties similar to those of these microorganisms.
Adlercreutzia equolifaciens subsp. celatus strain DSM 18785
Adlercreutzia equolifaciens subsp. equolifaciens strain DSM 19450
Bacteroides ovatus strain E-23-15
Bifidibacterium breve strain ATCC 15700
Bifidobacterium longum strain BB536
Clostridium sp. strain HGH136
Eggerthella sp. strain Julong 732
Eggerthella sp. strain YY7918
Eggerthella sp. strain D1
Enterococcus faecalis strain INIA P333
Enterococcus faecium strain EPI1
Enterohabdus mucosicola strain Mt1B8
Eubacterium sp. strain D2
Finegoldia magna strain EPI3
Lactobacillus fermentum strain DPPMA114
Lactobacillus intestinalis strain KTCT13676BP
Lactobacillus mucosae strain EPI2
Lactobacillus paracasei strain JS1
Lactobacillus plantarum strain DPPMA24W
Lactobacillus plantarum strain DPPMASL33
Lactobacillus rhamnosus strain DPPMAAZ1
Lactobacillus rhamnosus strain INIA P540
Lactobacillus sp. strain Niu-016
Lactococcus garvieae strain 20-92
Paraeggerthella sp. strain SNR40-432
Pediococcus pentosaceus strain CS1
Proteus mirabilis strain LH-52
Sharpea azabuensis strain ST18
Slackia equolifaciens strain DSM 24851
Slackia isoflavoniconvertens strain DSM 22006
Slackia sp. strain FJK1
Slackia sp. strain NATTS
Slackia sp. strain YIT11861
Slackia sp. strain TM-30
Streptococcus constellatus strain E-23-17
Streptococcus intermedius strain A6G-225
Veillonella sp. strain EP

Note that, each of the anaerobic microorganisms described above is available from the depository indicated in the accession number. The accession numbers each indicate that the anaerobic microorganism has been deposited in one of the following depositories.
FERM: International Patent Organism Depositary (IPOD)
http://unit.aist.go.jp/pod/ci/index.html
DSM: German Collection of Microorganisms and Cell Cultures (DSMZ)
http://www.dsmz.de/
KCCM: Korean Culture Center of Microorganisms

In an embodiment of the present invention, the anaerobic microorganism capable of producing equol is cultured under conditions suitable for the production of equol. The conditions suitable for the production of equol in an embodiment of the present invention refer to conditions under which the survival and activity of the anaerobic microorganism having the activity of producing equol are maintained. More specifically, the conditions refer to conditions under which the gas phase conditions (anaerobic conditions) in which the anaerobic microorganism can survive are maintained, and nutrients for supporting the activity and growth of the anaerobic microorganism are provided. There are various medium compositions known as suitable for the survival of anaerobic microorganisms. Therefore, those skilled in the art can select an appropriate medium composition for the above-described anaerobic microorganism capable of producing equol. Examples of the medium include, but are not limited to, a BHI medium available from Difco Laboratories, a medium used in Examples, and the like.

A water-soluble organic substance can be added as a carbon source to the medium used in an embodiment of the present invention. Examples of the water-soluble organic substance include, but are not limited to, the following compounds.
Sugars such as sorbose, fructose, and glucose;
Alcohols such as methanol;
Organic acids such as valeric acid, butyric acid, propionic acid, acetic acid, and formic acid, or salts thereof.

The concentration of the organic substance added to the medium as a carbon source can be adjusted as appropriate to efficiently grow the anaerobic microorganism in the medium.

A nitrogen source can also be added to the medium. In an embodiment of the present invention, various nitrogen compounds that may be used commonly in fermentation can be used as the nitrogen source. Preferred inorganic nitrogen sources are ammonium salts and nitrates. Examples of more preferred inorganic nitrogen sources include ammonium sulfate, ammonium chloride, ammonium phosphate, ammonium hydrogen phosphate, potassium nitrate, and sodium nitrate.

Meanwhile, examples of preferred organic nitrogen sources include amino acids, yeast extracts, peptones, meat extracts, liver extracts, and digested serum. Examples of more preferred organic nitrogen sources include arginine, cysteine, cystine, citrulline, lysine, yeast extracts, and peptones.

Furthermore, another organic substance or inorganic substance suitable for the production of equol can also be added to the medium in addition to the carbon source and the nitrogen source. For example, in some cases, the growth and activity of the anaerobic microorganism can be enhanced by adding, to the medium, a cofactor such as a vitamin or an inorganic compound such as various salts. Examples of plant- and animal-derived cofactors for microbial growth, such as inorganic compounds and vitamins, include the following.

| Inorganic Compounds | Vitamins |
|---|---|
| Potassium dihydrogen phosphate | Biotin |
| Magnesium sulfate | Folic acid |
| Manganese sulfate | Pyridoxine |
| Sodium chloride | Thiamine |
| Cobalt chloride | Riboflavin |
| Calcium chloride | Nicotinic acid |
| Zinc sulfate | Pantothenic acid |
| Copper sulfate | Vitamin B12 |
| Alum | Thioctic acid |
| Sodium molybdate | p-Aminobenzoic acid |
| Potassium chloride | |
| Boric acid, etc. | |
| Nickel chloride | |
| Sodium tungstate | |
| Sodium selenate | |
| Iron (II) ammonium sulfate | |

A commonly known method can be used as a method for adding these inorganic compounds, vitamins, or growth cofactors to produce a culture solution. The culture medium can be a liquid, a semi-solid, or a solid. In an embodiment of the present invention, the medium is preferably in the form of a liquid medium.

The medium according to an embodiment of the present invention may contain dextrins. When the anaerobic microorganism is cultured in a medium containing dextrins, a liquid containing equol and dextrins can be prepared without separately bringing dextrins into contact with the culture after culturing.

The addition of dextrins to the medium can be carried out before or during the culturing of the microorganism.

In the method according to an embodiment of the present application, the anaerobic microorganism can be cultured according to a known method of culturing anaerobic microorganisms. In industrial production, a continuous culture system (continuous fermentation system) that is capable of continuously feeding a medium and a gaseous substrate and that is provided with a mechanism for retrieving the culture can be used.

When the anaerobic microorganism is used in the production of the food composition containing the equol clathrate, oxygen is preferably prevented from entering the incubator. A commonly used fermenter can be used in itself as the incubator. An anaerobic atmosphere can be created by replacing oxygen mixed in the fermenter with an inert gas such as nitrogen.

Depending on the shape of the fermenter, a stirrer or the like can be used to sufficiently stir the medium. By stirring the culture in the fermenter, the chances of the anaerobic microorganism being in contact with the medium components and the gaseous substrate increases, which leads to optimized production efficiency of equol. The gaseous substrate can also be supplied as nanobubbles.

In the method according to an embodiment of the present application, the production of equol may be carried out in a closed system such as a bottle or a test tube sealed with a rubber stopper without gas supply. The production of equol can also be carried out in a mixed gas phase of one or more types of gas, including hydrogen. When the production of equol is carried out under a mixed gas phase, the hydrogen concentration is not limited.

When the method according to an embodiment of the present application is carried out in a mixed gas phase, the combination of gases constituting the gas phase is not limited, and one or more types of gas selected from carbon dioxide, nitrogen and the like may be used as a constituent component in addition to hydrogen. When gas is supplied to efficiently produce equol, the amount of the mixed gas constituting the gas phase supplied to the fermenter is preferably from 0.01 to 2.0 V/V/M (gas volume/liquid volume/minute).

In the method according to an embodiment of the present application, the microorganism can be cultured under normal pressure. When culturing the microorganism is performed under pressure, the pressurization conditions are not limited as long as the microorganism can grow. Examples of the pressurization conditions preferably include, but are not limited to, a pressure in a range from 0.02 to 0.2 MPa.

To increase the amount of equol produced, the temperature of the fermenter is not limited, but is preferably from 30° C. to 40° C., more preferably from 33° C. to 38° C., for example.

The fermentation time can be appropriately set according to the amount of equol produced, the remaining amount of isoflavones, and the like. Examples of fermentation time include, but are not limited to, 8 to 120 hours, preferably 12 to 72 hours, and particularly preferably 16 to 60 hours.

Before use, the fermented culture resulted from the culturing method according to an embodiment of the present invention can be turned into a solid form by a heat-drying process, a spray-drying process, or a freeze-drying process as necessary. The heat-drying process or the spray-drying process can be performed using, for example, a spray-drying device. The freeze-drying process can be performed using a freeze-drying apparatus. The heat-dried, spray-dried, or freeze-dried fermented culture may be subjected to a pulverizing process as necessary.

### EXAMPLES

The present invention will be described below based on examples, but the scope of the present invention is not limited to the following examples.

### Example 1

### <<Pre-culture>>

18-mm test tubes for culturing anaerobic microorganisms (available from Sanshin Industrial Co., Ltd.) were each filled with 10 mL of a medium having the composition shown in Table 1 and having been adjusted to pH 6.9. Then, butyl rubber stoppers and plastic caps were fitted to the test tubes while the gas phase was being purged with nitrogen, followed by sterilization at 115° C. for 15 minutes. Adlercreutzia equolifaciens subsp. celatus strain DSM 18785 was inoculated into this medium, and the gas phase was purged, for 2 minutes or longer, with hydrogen gas passed through a sterile filter. Then, shaking culture was performed at 37° C. and 200 spm for 18 hours, resulting in a pre-culture solution.

TABLE 1

| Material | Concentration | |
|---|---|---|
| Yeast extract | 10.0 | g/L |
| Peptone | 5.0 | g/L |
| Trypticase peptone | 5.0 | g/L |
| Glucose | 5.0 | g/L |
| Sodium chloride | 0.08 | g/L |
| Dipotassium hydrogen | 0.04 | g/L |
| phosphate | | |
| Monobasic potassium | 0.04 | g/L |
| phosphate | | |
| Magnesium sulfate | 0.02 | g/L |
| heptahydrate | | |
| Calcium chloride dihydrate | 0.01 | g/L |
| Sodium hydrogen carbonate | 0.4 | g/L |
| L-Cystine | 0.5 | g/L |

### <<Preparation of Equol>>

100-mL vials (available from Maruemu Corporation) were each filled with 20 mL of a medium having the composition shown in Table 1 with 3.3 g/L of daidzein, 15 g/L of β-cyclodextrin, and 10 g/L of L-arginine added and with the pH adjusted to 6.9. Then, butyl rubber stoppers (available from Maruemu Corporation) were fitted to the vials, and the gas phase was purged with nitrogen, followed by sterilization at 115° C. for 15 minutes. This medium was inoculated with 0.4 mL of the pre-culture solution prepared in Example 1, and the gas phase was purged, for 2 minutes or longer, with hydrogen gas passed through a sterile filter. Then, shaking culture was performed at 37° C. and 200 spm for 48 hours. After 48 hours, 2.8 g/L of equol was produced in the culture solution.

### <<Preparation of Equol Clathrate>>

The culture solution prepared in <<Preparation of Equol>> described above was heated at 80° C. for 10 minutes. After that, the culture solution was centrifuged at 6000 rpm for 10 minutes, and the supernatant was sterilized by filtration through a 0.2-μm membrane. The resulting sterilized solution was freeze-dried, resulting in a dried product A-1 containing an equol-β-cyclodextrin clathrate.

### <<Solubility Test>>

The dried product A-1 containing an equol clathrate prepared in <<Preparation of Equol Clathrate>> described above was subjected to a solubility test. As a control, a solubility test was performed on equol (available from LC Laboratories) alone.

Specifically, solubility tests were conducted under the following conditions.

300 mg of the dried product A-1 containing an equol clathrate was weighed and mixed with 10 mL of water. Then, the mixture was stirred at room temperature for 2 hours. This liquid was filtered through a 0.2-μm filter, resulting in a solution B-1 of the dried product A-1 containing an equol clathrate. The equol concentration of the solution B-1 was analyzed.

Meanwhile, 10 mg of equol (available from LC Laboratories) was weighed and subjected to the same operation, resulting in a liquid C-1 containing equol alone. The concentration of equol dissolved in the solution C-1 was analyzed.

According to the result, the concentration of equol in the solution B-1 was 1.9 mg/mL, whereas the equol concentration in the solution C-1 was 0.1 mg/mL, indicating that the equol clathrate A-1 has higher solubility than equol alone.

### <<Pharmacokinetics (PK)>>

The equol clathrate A-1 prepared in <<Preparation of Equol Clathrate>> was subjected to pharmacokinetics (PK) analysis using 6-week-old Crl:CD (SD) rats by oral administration. As a control, pharmacokinetics (PK) analysis of equol (available from LC Laboratories) alone was also performed.

Specifically, the administered dose of the equol clathrate A-1 was 100 mg/kg, whereas the dose of equol (available from LC Laboratories) was 6.3 mg/kg, an amount considered to be equivalent to 100 mg/kg of the equol clathrate A-1.

The administered dose of the equol clathrate A-1 and the administered dose of equol were each administered as a single oral dose to 3 animals/group. The test substance used for the administration sample was suspended in 0.5% CMC aqueous solution, and the administered volume was 10 mL/kg.

PK was measured using plasma, and blood was collected at 0 min, 30 min, 1 h, 2 h, 6 h, 12 h, and 24 h. After 24 hours of blood collection, the rats were euthanized.

No deaths or toxic symptoms were observed in this study. Further, at autopsy 24 hours after administration, no abnormality was observed in macroscopic observation of lymph nodes and the organs in the cranial cavity, thoracic cavity, and abdominal cavity.

The PK results are listed in Table 2. Note that, in Table 2, “Cmax” means maximum blood concentration, “Tmax” means time to reach Cmax, and “AUC0-24” means area.

It can be seen from Table 2 that the Cmax (maximum blood concentration) of the equol clathrate A-1 is at least twice as high as the Cmax (maximum blood concentration) of equol alone. In addition, it can be seen that the AUC0-24, or area, of the equol clathrate A-1 is higher than the AUC0-24, or area, of the equol alone. These results indicate that the equol clathrate according to an embodiment of the present invention has good equol absorbability.

TABLE 2

| | |
|---|---|
| Administered | Measured value |

| substance/administered dose | Cmax | Tmax | AUC0-24 |
|---|---|---|---|
| (mg/kg) | (ng/ml) | (h) | (ng · h · ml) |
| Equol clathrate/100 mg/kg | 86.04 | 1.0 | 579.69 |
| Equol/6.3 mg/kg | 41.64 | 0.5 | 362.93 |

### Example 2

### <<Preparation of Equol 2>>

Soybean hypocotyls (available from Fuji Oil Co., Ltd.) were pulverized using a Waring blender for five times and 1 minute each time. 27 g of the pulverized product was mixed with 0.27 g of pectinase G Amano (available from Amano Enzyme Inc.) and 118 g of deionized water. The mixture was then subjected to an enzymatic treatment while being stirred at 50° C. with the pH maintained at from 4 to 7.

Here, nutrient sources were added to give the mixture the composition shown in Table 1, L-arginine was added to reach an amount of 10 g/L, and the pH of the mixture was adjusted to from 6.8 to 7.0, resulting in a prepared solution. After that, 100-mL vials (available from Maruemu Corporation) were each filled with 20 mL of the prepared solution. Then, butyl rubber stoppers (available from Maruemu Corporation) were fitted to the vials, and the gas phase was purged with nitrogen, followed by sterilization at 115° C. for 15 minutes. This medium was inoculated with 0.4 mL of a pre-culture solution prepared in the same manner as in Example 1, and the gas phase was purged, for 2 minutes or longer, with hydrogen gas passed through a sterile filter. Then, shaking culture was performed at 37° C. and 200 spm for 48 hours.

After 48 hours, HPLC analysis confirmed the formation of equol.

This culture solution was heated at 80° C. for 10 minutes and then freeze-dried, resulting in a fermented soybean hypocotyl powder containing equol.

### <<Preparation of Equol Clathrate 2>>

1 g of the fermented soybean hypocotyl powder prepared in <<Preparation of Equol 2>> described above was supplemented with 10 mL of β-cyclodextrin 15 g/L aqueous solution, and mixed well. After that, the mixture was centrifuged at 6000 rpm for 10 minutes, and the supernatant was filtered through a 0.2-μm membrane. The filtrate was turned into a powder by freeze-drying, resulting in a dried product A-2 containing an equol clathrate.

In addition, 2 g of the fermented soybean hypocotyl powder prepared in <<Preparation of Equol 2>> described above was combined with Milli-Q water, and the same operation was performed, resulting in a dried product D-1.

### <<Solubility Test 2>>

The dried product A-2 containing an equol clathrate prepared in <<Preparation of Equol Clathrate 2>> described above was dissolved in Milli-Q water, resulting in a solution B-2.

As a control, the dried product D-1 was also dissolved in Milli-Q water, resulting in a solution C-2. The amount of Milli-Q water to be added was an amount allowing the dried product prepared in <<Preparation of Equol Clathrate 2>> to return to the original liquid amount.

The equol concentrations of the solutions B-2 and C-2 were measured by HPLC.

According to the result, the concentration of equol in the solution B-2 was 0.28 mg/mL, whereas the equol concentration in the solution C-2 was 0.02 mg/mL. It can be seen that the equol clathrate A-2 has higher solubility than the non-clathrate D-1.

1-9. (canceled)

10. A method for improving blood absorption of equol via oral ingestion, characterized by including the equol in β-cyclodextrin in the presence of water to form an equol-β-cyclodextrin inclusion complex.

11. The method according to claim 10, comprising administering the equol-β-cyclodextrin inclusion complex.

12. A method for improving blood absorption of equol in a subject, comprising administering an equol-β-cyclodextrin inclusion complex to a subject.
